SEQUENCE LISTING (5 sequences)

SEQ ID NO: 1 - DNA, 24 bp, Artificial sequence
  Primer RfmF
tcactaaagatcgagatagtacca

SEQ ID NO: 2 - DNA, 22 bp, Artificial sequence
  Primer RfmR
taacatcttcaacgtttcggtg

SEQ ID NO: 3 - DNA, 23 bp, Artificial sequence
  Primer of SCAR3 marker
tcactaaagatcgagatagtacc

SEQ ID NO: 4 - DNA, 22 bp, Artificial sequence
  Primer of SCAR3 marker
taacatcttcaacgtttcggtg

SEQ ID NO: 5 - DNA, 187 bp, Artificial sequence
  Rfm marker
tcactaaagatcgagatagtaccaaccaacatactagtttttgtactaattttcaaagcgtaaactttactgatcagcttccttactgagtagttattgcagccaagggaaacatgacaagtcagagggcttttgatctttgatctcttaccgttttcactctgccaccgaaacgttgaagatgtta

(19) United States
(12) Patent Application Publication
(10) Pub. No.: US 2013/0212727 A1
(43) Pub. Date: Aug. 15, 2013

(54) HYBRID CANOLA QUALITY BRASSICA JUNCEA

(71) Applicant: VITERRA, INC., (US)

(72) Inventors: Kening YAO, Saskatoon (CA); Laurie J. FRIESEN, Saskatoon (CA); Alan W. GROMBACHER, Saskatoon (CA); Thomas J. KUBIK, Saskatoon (CA); Chandra P. ANDRAHENNADI, Saskatoon (CA); Bruce J. HARRISON, Saskatoon (CA); Derek A. POTTS, Saskatoon (CA)

(73) Assignee: VITERRA, INC., Regina (CA)

(21) Appl. No.: 13/730,257

(22) Filed: Dec. 28, 2012

(30) Foreign Application Priority Data
Jan. 23, 2012 (CA) 2765353

(51) Int. Cl. C12N 15/82

(52) U.S. Cl. CPC C12N 15/8286; C12N 15/8274; C12N 15/8281; C12N 15/8283; C12N 15/8282; C12N 15/8247; C12N 15/8245
USPC 800/260; 435/419; 554/9; 426/635

(57) ABSTRACT

A seed from a first Brassica juncea oilseed plant comprising the cytoplasm of Moricandia arvensis and characterized as being cytoplasmic male sterile is provided. The seed from the Brassica juncea oilseed plant comprises canola quality oil and canola quality meal. A seed from a second Brassica juncea oilseed plant comprising a homozygous fertility restorer gene (Rfm) for mori cytoplasmic male sterility is also provided, wherein the Brassica juncea seeds have a canola quality oil and a canola quality meal. Also provided are hybrid Brassica juncea oilseed plants and methods to make hybrid Brassica juncea oilseed plants.

### FIELD OF INVENTION

The present invention relates to producing hybrid seeds of Brassica juncea. Furthermore this invention provides Canola quality seeds of B. juncea.

### BACKGROUND OF THE INVENTION

Hybrid vigor of higher plants, also known as heterosis, is often obtained through the use of cytoplasmic male sterility (CMS). The CMS is a maternally inherited trait that produces non functional pollen or no pollen at all. In order to produce hybrid seeds that are fertile, the system also needs a male plant that not only produces functional pollen but also has a fertility restorer gene in its genome.

Several hybrid systems have been developed for agriculturally important crops. In Brassica, for example, a number of CMS lines of various origins have been reported. For example, pol CMS in Brassica napus has been reported from spontaneous origin (Fu, 1981, Eucarpia Crucifer Newsletter 6: 6-7). Ogura CMS Brassica napus has been developed by cytoplasmic fusion with the male sterile radish ogura (Ogura 1968, Mem. Fac. Agric. Kagoshima Univ. 6: 39-78). Many other CMS systems have been developed.

Each system has its own disadvantages and limitations. For example, the pol CMS line is sensitive to environment in different nuclear backgrounds, leading to its breakdown and subsequent self pollination during the process of hybrid seed production. Ogura CMS Brassica napus is very stable, however, the restorer lines for the ogura CMS initially had very high glucosinolate levels because of the large radish chromosome fragment in the Brassica genome. Therefore, it has been known that the radish fragment contains genes that negatively impacts the level of glucosinolates in the B. napus restorer line. This represents a very significant quality trait of canola. For this reason, reducing the radish fragment without losing the restorer gene itself has been a main breeding goal around the world. Intensive efforts have been made to improve the restorer line, leading to developments of improved restorer lines with reduced glucosinolate contents (see for example CA 2714400; CA 2551781; CA 2283493 and CA 2303712). Because of these improvements, the ogura CMS system is currently a very popular hybrid system used in hybrid B. napus seed production in Canada, Australia and Europe.

Brassica napus and B. rapa were originally the only Brassica species that had been developed to produce canola oil. To be classified as canola, genotypes must have an erucic acid content of less than two percent in the oil and a glucosinolate content of less than 30 micromoles per gram of meal. B. juncea is grown in many countries of the world for the production of mustard and edible oil. Mustard and canola quality B. juncea are different genotypes as mustard quality genotypes of B. juncea are high in glucosinolate and erucic acid content. Breeding efforts have been made to develop canola quality B. juncea, and B. juncea germplasm has been developed that is low in glucosinolate (less than 30 umol/g dry seed weight) and in erucic acid content (less than 2% by weight). This germplasm is referred to as “canola quality” and is preferred for edible oil consumption (see for example U.S. Pat. No. 6,303,849, U.S. Pat. No. 7,605,301; CA 2,253,984; AU 2003204171).

The genetic relationship among the Brassica species was described as the “Triangle of U” (U, 1935, Jpn J. Bot. 7: 389-452). There are three diploid species, with the genome of B. rapa designated as ‘A’, the genome of B. nigra designated as ‘B’ and the genome of B. oleracea designated as ‘C’. There are three allotetraploid species in which the diploid genomes are combined. Thus, B. juncea has an ‘AB’ genomic constitution by combining the genomes of B. rapa and B. nigra, B. napus has the ‘AC’ genomes from B. rapa and B. oleracea, and B. carinata has the ‘BC’ genomes from B. nigra and B. oleracea. During meiosis, the chromosomes from each genome will pair with their homologues, thus in B. juncea, ‘A’ chromosomes will pair with ‘A’ and ‘B’ will pair with ‘B’. Interspecific crosses can be made between Brassica species, but progeny of the cross will be sterile. In a cross between B. juncea and B. napus, for example, the common ‘A’ chromosomes will pair, but the ‘B’ and ‘C’ chromosomes will not pair well, causing sterility. Crossing back to either species can restore fertility, but the alien genome chromosomes are lost. For this reason, it is very difficult to get genetic transfer between chromosomes of different genomes, for example from the ‘C’ genome of B. napus to the ‘B’ genome of B. juncea.

The radish fragment bearing the ogura CMS fertility restorer gene is know to be located in the ‘C’ genome to the B. napus. For this reason, it has been very difficult to transfer the ogura CMS fertility restorer gene from B. napus to B. juncea through inter-specific crossing. Even when the radish fragment has been successfully incorporated into the genome of canola quality B. juncea, the resulting seed has a brown seed coat; a trait that is not acceptable for canola quality juncea. Recently, a canola quality B. juncea hybrid variety, 45J10, was developed using the ogura CMS system and the variety was registered in Canada (Canadian Food Inspection Agency, plant variety database). There are problems with this variety. For example, there is a enlarged roots associated with this hybrid juncea. This enlarged root phenomenon is also known as hybridization nodules. Indeed, there has never been a significant commercial seed sale for this variety. Therefore, there is still a need to develop other CMS systems for canola quality B. juncea hybrid seed.

A cytoplasmic male sterility system has been developed in mustard following repeated backcrosses of a somatic hybrid between Moricandia arvensis and B. juncea with mustard B. juncea (Prakash et al., 1998, Theor Appl Genet 97: 488-492). The somatic hybrid contains mitochondria and chloroplast from Moricandia arvensis (Kirti et al., 1992, Plant cell rep 11: 318-321). A B. juncea fertility restorer line (Rfm) for the CMS (mori) lines has also been established (Prakash et al., 1998, Theor Appl Genet 97: 488-492). Ten years after the novel CMS (mori) and fertility restoration system were developed, the National Research Centre on Rapeseed Mustard released a hybrid mustard (B. juncea) variety, NRCHB 506, which was the first commercial hybrid variety based on the Moricandia CMS system. However, a hybrid B. juncea has only been demonstrated in mustard juncea which has high glucosinolates, high erucic acid and low oleic acid therefore it can not be used and marketed as canola. It is not known whether the mori CMS system can be used in canola quality B. juncea.

### SUMMARY OF THE INVENTION

The present invention relates to hybrid Brassica juncea and to Canola quality seeds of B. juncea.

It is an object of the invention to provide an improved hybrid Canola quality Brassica juncea.

According to the present invention there is provided a seed from a Brassica juncea oilseed plant comprising the cytoplasm of Moricandia arvensis, the Brassica juncea oil seed plant characterized as being cytoplasmic male sterile, and the seed comprising canola quality oil and canola quality meal. The seed may comprise less than 30 umole per gram oil-free total glucosinolates, less than 2% erucic acid by weight and more than 55% oleic acid by weight. The seed may be obtained from progeny of a cross between the Brassica juncea line AM-J05Z-10367 that has been deposited Nov. 17, 2011, and has a ATCC accession No. PTA-12261, and any canola quality Brassica juncea line.

The present invention also provides a Brassica juncea plant, or any progeny thereof, that produces a seed comprising the cytoplasm of Moricandia arvensis, the Brassica juncea oil seed plant characterized as being cytoplasmic male sterile, and the seed comprising canola quality oil and canola quality meal. The seed may comprise less than 30 umole per gram oil-free total glucosinolates, less than 2% erucic acid by weight and more than 55% oleic acid by weight. The plant may be obtained from progeny of a cross between the Brassica juncea line AM-J05Z-10367 that has been deposited Nov. 17, 2011, and has a ATCC accession No. PTA-12261, and any canola quality Brassica juncea line.

The present invention also provides a seed from an Brassica juncea oilseed plant comprising a homozygous fertility restorer gene (Rfm) for mori cytoplasmic male sterility, wherein the Brassica juncea seeds have an oil and meal in canola quality. The seed may comprise less than 30 umole per gram oil-free total glucosinolates, less than 2% erucic acid by weight, and more than 55% oleic acid by weight. The seed may be obtained from the Brassica juncea line JM0Z-909643 that has been deposited Nov. 17, 2011, and has a ATCC accession No. PTA-12260, or the seed may be obtained from progeny of a cross between the Brassica juncea line JM0Z-909643 that has been deposited Nov. 17, 2011, and has a ATCC accession No. PTA-12260, and any canola quality Brassica juncea plant or any canola quality Brassica juncea plant comprising the cytoplasm of Moricandia arvensis.

The present invention also pertains to a Brassica juncea plant, or any progeny thereof, that produces a seed comprising a homozygous fertility restorer gene (Rfm) for mori cytoplasmic male sterility, wherein the Brassica juncea seed comprises an canola quality oil and a canola quality meal. The seed may comprise less than 30 umole per gram oil-free total glucosinolates, less than 2% erucic acid by weight, and more than 55% oleic acid by weight. The plant, or any progeny thereof, may be derived from the Brassica juncea line JM0Z-909643 that has been deposited Nov. 17, 2011, and has a ATCC accession No. PTA-12260.

The present invention also includes a plant cell derived from a Brassica juncea oilseed plant deposited under ATCC accession No. PTA-12260, Nov. 17, 2011, progeny of a plant cell derived from a Brassica juncea oilseed plant deposited under ATCC accession No. PTA-12260, a Brassica juncea oilseed plant deposited under ATCC accession No. PTA-12261, Nov. 17, 2011, progeny of a plant cell derived from a Brassica juncea oilseed plant deposited under ATCC accession No. PTA-12261, or a combination thereof.

The present invention also provides a method for making hybrid Brassica juncea seed comprising, crossing a first Brassica juncea plant comprising the cytoplasm of Moricandia arvensis, the first Brassica juncea oil seed plant characterized as being cytoplasmic male sterile, with a second Brassica juncea plant comprising a homozygous fertility restorer gene (Rfm) for mori cytoplasmic male sterility, to produce the hybrid Brassica juncea seed, the hybrid Brassica juncea seed characterized as canola quality and comprising a hybridity level of at least of 90%. The hybrid Brassica juncea seed may comprise less than 30 umole per gram oil-free total glucosinolates, less than 2% erucic acid by weight, and more than 55% oleic acid by weight.

The present invention also provides hybrid Brassica juncea plant arising from a cross of a first Brassica juncea plant comprising the cytoplasm of Moricandia arvensis, the first Brassica juncea oil seed plant characterized as being cytoplasmic male sterile, with a second Brassica juncea plant comprising a homozygous fertility restorer gene (Rfm) for mori cytoplasmic male sterility, the hybrid Brassica juncea plant capable of producing seed comprising less than 30 umole per gram oil-free total glucosinolates, less than 2% erucic acid by weight, and more than 55% oleic acid by weight.

The present invention includes a crushed seed meal-oil composition obtained by crushing a seed from Brassica juncea oil seed plant comprising cytoplasm of Moricandia arvensis, the Brassica juncea oil seed plant characterized as being cytoplasmic male sterile, and the seed comprising canola quality oil and canola quality meal. The crushed seed meal-oil composition may comprise less than 30 umole per gram oil-free total glucosinolates, less than 2% erucic acid by weight, more than 55% oleic acid by weight, or a combination thereof. Furthermore, the seed may be obtained from progeny of a cross between the Brassica juncea line AM-J05Z-10367 (that has been deposited Nov. 17, 2011, and has a ATCC accession No. PTA-12261), and any canola quality Brassica juncea line.

The present invention also provides a plant cell derived from a Brassica juncea plant that produces a crushed seed meal-oil composition comprising cytoplasm of Moricandia arvensis, the Brassica juncea oil seed plant characterized as being cytoplasmic male sterile, and comprising less than 30 umole per gram oil-free total glucosinolates, less than 2% erucic acid by weight, more than 55% oleic acid by weight, or a combination thereof. The crushed seed may be obtained from seed obtained from progeny of a cross between the Brassica juncea line AM-J05Z-10367 (that has been deposited Nov. 17, 2011, and has a ATCC accession No. PTA-12261), and any canola quality Brassica juncea line.

The present invention also provides a use of a crushed seed meal-oil composition obtained by crushing a seed from Brassica juncea oil seed plant comprising cytoplasm of Moricandia arvensis, the Brassica juncea oil seed plant characterized as being cytoplasmic male sterile, for preparing an oil comprising less than 30 umole per gram oil-free total glucosinolates, less than 2% erucic acid by weight, and more than 55% oleic acid by weight. The crushed seed meal may be obtained seed obtained from progeny of a cross between the Brassica juncea line AM-J05Z-10367 (that has been deposited Nov. 17, 2011, and has a ATCC accession No. PTA-12261), and any canola quality Brassica juncea line.

The present invention includes a method of obtaining an oil comprising less than 30 umole per gram oil-free total glucosinolates, less than 2% erucic acid by weight, and more than 55% oleic acid by weight, the method comprising:

growing a plant produced from a seed from a cytoplasmic male sterile Brassica juncea oil seed plant comprising cytoplasm of Moricandia arvensis;

obtaining progeny seed from the plant; and

extracting the oil from the progeny seed obtained from the plant.

In the step of growing, in the method as just described, the seed may be a Brassica juncea oil seed plant deposited under ATCC No. PTA-12260, Nov. 17, 2011, or PTA-12261, Nov. 17, 2011.

The present invention also provides a method of obtaining an oil comprising less than 30 umole per gram oil-free total glucosinolates, less than 2% erucic acid by weight, and more than 55% oleic acid by weight, the method comprising:

growing a plant obtained from progeny of a cross between a Brassica juncea oil seed plant comprising the cytoplasm of Moricandia arvensis, the Brassica juncea oil seed plant characterized as being cytoplasmic male sterile, and producing seed comprising canola quality oil and canola quality meal, and any canola quality Brassica juncea line;

obtaining seed from the progeny; and

extracting the oil from the seed from the progeny.

In the step of growing, in the method as just described, the seed may be a Brassica juncea oil seed plant deposited under ATCC No. PTA-12261, Nov. 17, 2011, and the canola quality B. juncea line may be PTA-12261, Nov. 17, 2011.

The present invention also provides a set of nucleic acids comprising the sequence of SEQ ID NO:1 and SEQ ID NO:2.

As described herein, the mori CMS system has been adapted in canola quality B. juncea hybrid seed production. Furthermore, various improvements have been made in terms of seed quality. Cytoplasmic male sterile (CMS) Brassica juncea plants with canola quality of oil and meal are provided. The CMS (mori) trait introgression was achieved by crossing canola quality B. juncea with the mustard CMS (mori) line, followed by extensive backcrossing, for example, up to 6 generations (BC6) with canola quality B. juncea germplasm.

Canola quality B. juncea lines with the homozygous fertility restorer gene for the CMS (mori) are also provided. The origin of the Rfm gene is from Moricandia arvensis, which was first introgressed into mustard (B. juncea). The canola quality Brassica juncea restorer lines obtained the fertility restorer gene (Rfm) from a mustard restorer line by crossing, producing doubled haploid plants and subsequent selection of traits such as erucic acid, glucosinolate profile, oleic acid and the Rfm gene with molecular markers. Until the present invention, the size of the Moricandia genome fragment was not known nor was it understood how this fragment would affect the quality traits of canola quality B. juncea.

As described herein, we demonstrate that the Moricandia arvensis fragment harboring the fertility restorer gene (Rfm) does not have negative effects on quality traits of canola quality B. juncea.

The present invention also provides hybrid seeds, produced using the CMS (mori) and restorer lines under field conditions. The hybrid seeds thus produced may be characterized as being canola quality. Levels of hybridity of the harvested hybrid seeds can be above 90%. In some cases, the hybridity level can be 100%.

The present invention also relates to parts of the canola quality Brassica juncea plant described herein, regardless of inbred or hybrid. The plant parts may be selected from a group of nucleic acid sequences (RNA, mRNA, DNA, cDNA), tissue, cells, pollen, ovules, roots, leaves, oilseeds, microspores and vegetative parts, whether mature or embryonic.

The Brassica plants of this invention may be used to breed a novel Brassica line. Therefore, the present invention also includes progeny derived from the Brassica plants and seeds described herein. The progeny may be obtained using isolation and transformation, conventional breeding, pedigree breeding, crossing, self-pollination, doubled haploidy, single seed descent and backcrossing.

The present invention also relates to canola quality Brassica juncea cytoplasmic male sterile (CMS) plants, wherein the plants have mitochondria and chloroplasts from Moricandia (Mori). The CMS (Mori) line produces sterile pollen, which can be used for hybrid seed production. Furthermore, canola quality Brassica juncea comprising a homozygous fertility restorer gene for mori cytoplasmic male sterility; i.e. the restorer line (Rfm) are also provided. The invention further relates to hybrid canola quality Brassica juncea plants that are produced by cross pollination of the said Brassica juncea CMS (Mori) lines with the Brassica juncea restorer lines.

Both Brassica juncea CMS (Mori) lines and restorer lines (Rfm) of the present invention are canola quality. These lines have less than 2% erucic acid by weight and less than 30 μmol/g glucosinolates on an oil-free dry seed meal weight basis. Hybrid seeds produced using these lines as parents are also canola quality having less than 2% erucic acid by weight and less than 30 μmol/g glucosinolates on an oil-free dry seed meal weight basis. Furthermore, these lines described herein have been developed to have a fatty acid profile with greater than 55% oleic acid as may be required for canola variety registration, for example in Canada.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a nucleotide sequence of the marker (SEQ ID NO:5) linked to the Rfm gene. Sequence specific PCR primers RfmF and RfmR were designed according to the nucleotide sequences marked as bold.

### DETAILED DESCRIPTION

The following description is of a preferred embodiment.

Canola quality Brassica juncea as used herein refers to B. juncea that produces seeds with oil and meal quality that meets the requirements for a commercial designation as canola oil or canola meal. Canola quality requires less than 30 μmole/g total glucosinolates on an oil-free dry seed meal basis, less than 2% erucic acid by weight and more than 55% oleic acid by weight (see CA 2,253,984 or U.S. Pat. No. 6,787,686 for a description of canola quality oil). Examples of canola quality B. juncea that may be used as described herein include but are not limited to XCEED 8570, XCEED 8571, XCEED Oasis CL, Estlin, Emulet, Arid, and J05Z-10367 (all available from Viterra Inc, Saskatoon, Sask Canada), J96D-2250 (ATCC 203101; described in CA 2,253,984 and U.S. Pat. No. 6,787,686), J96D-2990 (ATCC 203102 (described in CA 2,253,984 and U.S. Pat. No. 6,787,686), J96D-0758 (ATCC 203103, described in CA 2,253,984 and U.S. Pat. No. 6,787,686), and ATCC PTA-12260 (described herein). Examples of canola quality B. juncea varieties that are currently marketed in Australia that may be used as described herein include but are not limited to Dune and Sahara CL (also available from Viterra Inc., Viterra Australia Research Farm, via courier: Grains Innovation Park, 110 natimuk Road, Horsham, Vic 3401, Australia). Each of these B. juncea varieties may be crossed with plants produced from the seeds of ATCC PTA-12261 as described herein.

Prior art mustard B. juncea has greater than 30 μmole/g total glucosinolates on an oil-free dry seed meal basis, greater than 2% erucic acid by weight and less than 55% oleic acid by weight, and is not designated as canola quality.

The term “line” refers to a group of plants that display no phenotypic variation among individuals sharing this designation.

A “variety” or “cultivar” is a line that may be used for commercial production.

A “doubled haploid” line or “DH line” refers to a line created by the process of microspore embryogenesis, in which a plant is created from an individual haploid microspore. During this tissue culture process, the chromosome numbers are doubled for example, by incubating with a chemical that doubles the chromosome content without accompanying cell division, for a period of time in a culture medium containing a carbon source. For example a haploid microspore may be incubated with 0.0031% colchicine for 48 hr at 34° C. in a culture medium containing 17% sucrose. This yields a plant with a diploid number of chromosomes where each chromosome pair is comprised of two duplicated chromosomes. Therefore, a DH line normally displays little or no genetic variation between individuals for traits and no segregation of traits in subsequent generations and lines are created that are homogeneous (i.e. all plants within the line have the same genetic makeup). The original DH plant is referred to as DH1, while subsequent generations are referred to as DH2, DH3 etc. Doubled haploid procedures are well known in the art and have been established for several crops. A procedure for B. juncea has been described by Thiagrarajah and Stringham (1993, A comparison of genetic segregation in traditional and microspore-derived populations of Brassica juncea in: L. Czern and Coss. Plant Breeding 111:330-334).

The fatty acid composition of oils may be determined by techniques known to one f skill in the art. For example, as described herein, fatty acid methyl esters were analyzed by GLC after hydrolysis of etherified fatty acids from its glycerol backbone. For example, 20 seeds from each DH line were homogenized in 2 ml of 0.5 N sodium methoxide in methanol and 1 ml of hexane that contained 500 μg of tripentadecanoin (TAG, G-15:0; Sigma). After adding 1 ml of distilled water, the homogenate was centrifuged for 5 min at 3500 rpm using a bench top centrifuge (Baxter Canlab Megafuge 1.0, Heraeus Instruments). 200 ul of the top layer was transferred into an auto-sampler vial and 900 ul of hexane was added into each vial. 2 ul of this sample was injected into the gas-liquid chromatography (GLC; Hewlett Packard Model 5890), which was equipped with a DB-23 column (0.25 mm id×30 m; Hewlett Packard) and flame ionization detector. The GLC was operated with the injector and detector temperatures at 250° C. and 300° C., respectively. The column temperature was initially held at 160° C. for 0.5 min and gradually increased to 245° C. at the rate of 10° C./min, and then held at 245° C. for 4 min. Helium was used as a carrier gas with flow rate of 1 ml/min. The eluted fatty acid methyl esters were integrated. The identify of each peak was confined by comparison with the following authentic standards (Sigma): palmitic acid (16:0), palmitoleic acid (16:1), stearic acid (18:0), oleic acid (18:1, Δ9), cis-vaccenic acid (18:1, Δ11), linoleic acid (18:2), linolenic acid (18:3), eicosanoic acid (20:0), cis-11 eicosenoic acid (20:1), cis-11, 14 eicosadinoic acid (20:2), docosanoic acid (22:0), erucic acid (22:1), cis-13, 16 docosadienoic acid (22:2), tetracosanoic acid (24:0) and cis-15 tetracosenoic acid (24:1). Each fatty acid is expressed as percentage of total fatty acids by weight.

Glucosinolate content may be measured by gas-liquid chromatography for quantification of trimethylsilyl (TMS) derivatives of extracted and purified desulfoglucosinolates, as described by Daun and McGregor (1981, Glucosinolate analysis of rapeseed <canola>, Method of the Canadian Grain Commission, Grain Research Laboratory). Benzyl glucosinolate was added to each sample as an internal control. Each glucosinolate peak was compared with the internal control and expressed as μmol. Total glucosinolate contents are the sum of each individual, and to expressed as μmol/g oil-free dry seed meal weight basis.

As used herein, “progeny” means one or more direct descendants, one or more indirect descendants, one or more offspring, one or more derivatives or a combination thereof, of a plant or plants described herein. Progeny may also include a first, second, third or subsequent generation plant, and may be produced by self crossing, crossing with plants with the same or different genotypes, and may be modified by range of suitable genetic engineering techniques.

In this application “breeding” includes all methods of developing or propagating plants and includes both intra species, inter species, intra line crosses, inter line crosses, and any suitable artificial breeding techniques. Desired traits may be transferred to other Brassica juncea lines through conventional breeding methods and can also be transferred to other Brassica species, such as Brassica napus and Brassica rapa through inter-specific crossing. Conventional breeding methods and inter-specific crossing methods as well as other methods of transferring genetic material between plants are well documented in the literature.

The term “backcross” refers to a cross of first-generation hybrid, F₁, with one parent or individual genetically identical to one of the two parents. In turn, the term “backcrossing” refers to a process of creating a backcross. In this application “inter-specific cross” means a cross made between two different species within the same genus. For example, a cross made between Brassica juncea and Brassica napus is designated as “inter-specific cross”. The term “self” means a plant is self-pollinated.

In this application “molecular biological techniques” means all forms of manipulation of a nucleic acid sequence to alter the sequence and expression thereof and includes the insertion, deletion or modification of sequences or sequence fragments and the direct introduction of new sequences into the genome of an organism by directed or random recombination using any suitable vectors and/or techniques.

In this application “genetically derived” as used for example in the phrase “genetically derived from the parent lines” means that the characteristic in question is dictated wholly or in part by an aspect of the genetic makeup of the plant in question.

In this application the term “Brassica” may comprise any or all of the species subsumed in the genus Brassica including Brassica napus, Brassica juncea, and Brassica rapa.

“Polymorphism” in a population refers to a condition in which the most frequent variant (or allele) of a particular locus has an allele frequency in the population which does not exceed 99%.

The term “heterozygosity” (H) is used when a fraction of individuals in a population have different alleles at a particular locus (as opposed to two copies of the same allele). Heterozygosity is the probability that an individual in the population is heterozygous at the locus. Heterozygosity is usually expressed as a percentage (%), ranging from 0 to 100%, or on a scale from 0 to 1.

“Homozygosity” or “homozygous” indicates that a fraction of individuals in a population have two copies of the same allele at a particular locus. Where plants are doubled haploid derived, it is presumed that subject to any spontaneous mutations occurring during duplication of the haplotype, all loci are homozygous. Plants may be homozygous for one, several or all loci as the context indicates.

The invention in part provides B. juncea plants which are capable of producing seeds that can be described as canola quality. For example the B. juncea plants are canola quality B. juncea cytoplasmic male sterile (CMS) plants, wherein the plants have mitochondria and chloroplast from Moricandia (mori). The CMS (Mori) line produces sterile pollen, and can be used for hybrid seed production. Although the CMS (mori) trait was transferred from mustard juncea, the mustard juncea genome was substantially replaced by canola quality B. juncea through repeated backcrossing followed by selection of canola quality traits such as glucosinolate content, erucic acid and oleic acid levels etc and other agronomic traits such as plant height, flowering time, seed set, silique length and yield etc.

The present invention in part provides a CMS (mori) B. juncea line that was developed by crossing mustard B. juncea with canola quality B. juncea lines and is referred herein as CMS (mori) canola quality B. juncea. One representative, non-limiting example of a line of this group is AM-J05Z-10367. In one example, the mustard CMS (mori) B. juncea line, MoriS, was used as the donor of cytoplasm, and the canola quality B. juncea variety, Estlin and canola quality B. juncea breeding line J05Z-10367, were used as nuclear genome donors. After 8 generations of backcrossing with Estlin and J05Z-10367, CMS (mori) B. juncea lines with canola quality were developed, as represented by the line AM-J05Z-10367. Also as described herein, a gradual and continuous improvement from mustard juncea into canola quality juncea has been observed (see for example Table 1).

Therefore, the present invention provides a seed from a Brassica juncea oilseed plant comprising the cytoplasm of Moricandia arvensis, the Brassica juncea oil seed plant characterized as being cytoplasmic male sterile, and the seed comprising canola quality oil and canola quality meal. The seed may comprise less than 30 umole per gram oil-free total glucosinolates, less than 2% erucic acid by weight and more than 55% oleic acid by weight. The seed may be obtained from progeny of a cross between the Brassica juncea line AM-J05Z-10367 that has been deposited Nov. 17, 2011, and has a ATCC accession No. PTA-12261, and any canola quality Brassica juncea line.

Non limiting examples of a canola quality B. juncea that may be crossed with plants derived from ATCC PTA-12261 include Oasis CL, XCEED 8570, XCEED 8571, Arid, Amulet, Estlin, J05Z-10367 (all available from Viterra Inc, Saskatoon, Sask Canada), J96D-2250 (ATCC 203101; described in CA 2,253,984 and U.S. Pat. No. 6,787,686), J96D-2990 (ATCC 203102 (described in CA 2,253,984 and U.S. Pat. No. 6,787,686), J96D-0758 (ATCC 203103, described in CA 2,253,984 and U.S. Pat. No. 6,787,686), and ATCC PTA-12260 (described herein). Other examples of canola quality B. juncea varieties that are currently marketed in Australia that may also be crossed with plants derived from ATCC PTA-12261 as described herein include but are not limited to Dune and Sahara CL (also available from Viterra Inc., Viterra Australia Research Farm, via courier: Grains Innovation Park, 110 natimuk Road, Horsham, Vic 3401, Australia).

The present invention also provides canola quality Brassica juncea line JM0Z-909643. The line JM0Z-909643 comprises a homozygous fertility restorer gene for mori cytoplasmic male sterility and therefore herein is referred to a restorer line. In one example (see Example 2), the mustard B. juncea line, MoriR4, was used as the donor of fertility restorer gene for mori CMS (the Rfm gene). However, canola quality B. juncea lines may also be used as donors of genes controlling the canola quality traits including glucosinolate and fatty acid profiles. DH lines as described herein were produced from advanced backcrosses (see for example Table 2). Line JM0Z-909643 is a non limiting example of a fertility restorer line for mori CMS with canola quality.

A deposit of seeds from lines AM-J05Z-10367 (ATCC designation: PTA-12261) and JM0Z-909643 (ATCC designation: PTA-12260) were made with the patent depository of the American Type Culture Collection (ATCC), Mansassas, Va. 20110-2209, USA.

Therefore, the present invention also provides a seed from an Brassica juncea oilseed plant comprising a homozygous fertility restorer gene (Rfm) for mori cytoplasmic male sterility, wherein the Brassica juncea seeds have an oil and meal in canola quality. The seed may comprise less than 30 umole per gram oil-free total glucosinolates, less than 2% erucic acid by weight, and more than 55% oleic acid by weight. The seed may be obtained from the Brassica juncea line JM0Z-909643 that has been deposited Nov. 17, 2011, and has a ATCC accession No. PTA-12260, or the seed may be obtained from progeny of a cross between the Brassica juncea line JM0Z-909643 that has been deposited Nov. 17, 2011, and has a ATCC accession No. PTA-12260, and any canola quality Brassica juncea plant or any canola quality Brassica juncea plant comprising the cytoplasm of Moricandia arvensis.

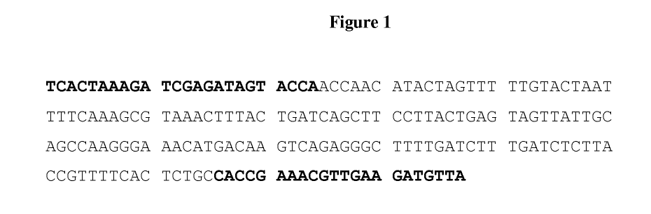

The present invention, also provides an improved genetic marker associated with the Rfm gene. The original SCAR3 marker (Prakash et al., 1998 Theor Appl Genet 97: 488-492) is not stable and not clear. The original primers of SCAR3: 5′-TCACTAAA GATCGAGATAGTACC-3′ (SEQ ID NO:3) and 5′-TAACATCTTCAACGTTTC GGTG-3′ (SEQ ID NO:4), did not produce both 400 bp and 200 bp fragments as expected. Their use resulted in the production of one 200 bp fragment. We therefore cloned and sequenced the DNA fragment. Sequence information was disclosed as shown in FIG. 1. Based on this sequence, specific primers, RfmF (5′-TCACTAAAGATCGAGATAGTACCA-3′; SEQ ID NO:1) and RfmR (5′-TAACATCTTCAACGTTTCGGTG; SEQ ID NO:2), were designed, tested and were used. These primer sequences result in a more robust and reliable screening marker. Using this new marker, we found no recombination between Rfm marker and Rfm gene has been found. This marker, as described in example 3, has been used for screening the presence of Rfm gene for all the potential fertility restorer DH lines.

When the Moricandia arvensis genome fragment was introgressed into mustard juncea, a restorer line was created, which restores mori CMS mustard juncea. The size of the Moricandia arvensis genome fragment was not determined (Prakash, 1998, Theor Appl Genet 97: 488-492; Sharma et al., 2007, Theor Appl Genet 114: 385-392). The effects of this Moricandia arvensis genome fragment on oil and meal quality can not be determined because of the mustard juncea genetic background (high glucosinolate and high erucic acid). The present invention demonstrates that when the Moricandia arvensis genome fragment was introgressed into canola quality B. juncea, it has no negative impact on canola quality, for example on the level of glucosinolates.

Glucosinolates are sulfur-based compounds that remain in the solid component of the seed, which is the solid meal left behind after the seed has been ground and its oil extracted. Their structure includes glucose in combination with aliphatic hydrocarbons (3-butenyl glucosinolate, 4-pentenyl glucosinolate, 2-hydroxy-3-butenyl glucosinolate, and 2-hydroxy-4-pentenyl glucosinolate) or aromatic hydrocarbons (3-indoylmethyl glucosinolate, 1-methoxy-3-indoyl methyl glucosinolate). Aliphatic glucosinolates are also known as alkenyl glucosinolates. Aromatic glucosinolates are also known as indoles.

High levels of glucosinolates are undesirable because they produce toxic by-products when acted upon by the enzyme myrosinase. Myrosinase is a naturally occurring enzyme present in Brassica species. When Brassica seed is crushed, myrosinase is released and catalyzes the breakdown of glucosinolates to produce glucose, thiocyanates, isothiocyanate and nitriles. When separated from glucose, these other products are toxic to certain mammals. Since solid meal is used as a livestock feed, the level of total glucosinolates should be less than 30 μmole/g of oil free dry seed meal basis in order to qualify for canola quality.

The Rfm gene, located in a genome fragment of Moricandia arvensis, was transgressed into mustard B. juncea to develop the fertility restorer (mori) line, MoriR4. The radish fragment in the early restorer lines for ogura CMS B. napus had shown a linkage between high glucosinolate level and the restorer gene (Delourme et al., 1998, Theor Appl Genet 97: 129-134; Delourme et al., 1999, In Proc 10^{th} Int Rapeseed Congr. Can berra, pp 26-29) Extensive breeding efforts were made by breeders to break the linkage in order to reduce the glucosinolate levels (Delourme et al., 1998, Theor Appl Genet 97: 129-134; Primard-Brisset et al., 2005, Theor Appl Genet 111: 736-746). Therefore, it was not known whether the Moricandia arvensis fragment would have negative effects on seed quality such as the level of glucosinolates. The effect of the Moricandia arvensis fragment can not be examined using a mustard quality B. juncea genetic background which already comprises high levels of glucosinolate.

The present invention demonstrates that all the undesired mustard B. juncea quality traits, such as seed color, levels of erucic acid and oleic acid, and levels of glucosinolates, can be segregated independently from the fertility restorer gene (Rfm gene). These criteria are import in order for the mori CMS system to be adapted to canola quality B. juncea hybrid production.

Quality analysis by gas chromatography confirmed that both Brassica juncea CMS (Mori) lines and restorer lines (Rfm) are canola quality. These lines have less than 2% erucic acid by weight and less than 30 μmol/g glucosinolate on an oil-free dry seed meal weight basis. As such, the hybrid seeds produced using these lines as parents are also canola quality in terms of erucic acid and oleic acid contents and glucosinolates levels.

The invention further provides hybrid seeds and hybrid seed production. As described herein, the mori CMS system has been successfully adapted to hybrid seed production for canola quality B. juncea. As demonstrated in real field conditions, seeds can be produced by cross pollination of the B. juncea CMS (Mori) lines with the B. juncea restorer lines. Level of hybridity of hybrid seeds produced in the field may be determined by testing the presence of the Rfm gene in the F1 hybrid seeds. As described below, levels of hybridity ranged from 90% to 100%, or any amount therebetween, when the restorer line JM0Z-909643 was used as father line with multiple mori CMS lines, including AM-J05Z-10367. For example, the level of hybridity of hybrids made from the combination of AM-J05Z-10367 and JM0Z-909643 is 97.5%.

The availability of mori CMS lines, representative line AM-J05Z-10367 (ATCC Accession No. PTA-12261, Nov. 17, 2011) and mori CMS fertility restorer lines, representative line JM0Z-909643 (ATCC Accession No. PTA-12260, Nov. 17, 2011) make it possible in the future to make canola quality B. juncea hybrid seeds using the mori CMS system through conventional plant breeding and molecular marker assisted selection (MAS) techniques, which are known to those skilled in the art.

Generating inbred plants using both mori CMS B. juncea lines and fertility restorer lines can be accomplished by using the plants of the present invention as crossing parents through known plant breeding and other associated techniques. Ideally, parent lines selected by the plant breeder are also canola quality so that the main focus of the crossing program will be the introduction of the CMS trait and Rfm gene to the other B. juncea plants. For example, the homozygous fertility restorer gene, Rfm, of the JM0Z-909643 plant can be introduced into other Brassica juncea plants or inbred lines by crossing and repeated backcrosses of the Brassica juncea plants. For selection of each backcrossing generation, the presence of the Rfm gene should be monitored using the markers disclosed in the invention. Similarly, the mori CMS trait can be introduced from AM-J05Z-10367 to other B. juncea plants by crossing and repeated backcrossing.

As described herein, all major traits in B. juncea related to canola quality, such as total glucosinolates content, erucic acid and oleic acid, can segregate properly. The Rfm gene can also segregate independently from any non-canola mustard quality traits. There is no negative linkage with the Rfm gene locus, although the size of the integrated Moricandia arvensis genome fragment flanking the Rfm gene locus is not known.

Generating hybrid seeds, plants, or a combination thereof may be carried out using the mori CMS and fertility restorer lines, or any other inbred lines that are progenies of the lines provided in the current invention. Brassica plants may be regenerated from the mori CMS B. juncea lines and fertility restorer lines of this invention using known techniques as described above.

Yield potential of the initial B. juncea hybrid lines made by the mori CMS system described herein was evaluated. The hybrid lines yielded from 101% to 107% in comparison with the open pollinated varieties. This indicates that the mori CMS hybrid system can not only be used for making canola quality B. juncea hybrid varieties, but also the varieties thus made have a potential yield increase. It is reasonable to predict that, by using the mori CMS hybrid system in canola quality B. juncea, increased heterosis potential can be achieved using diversified genetic backgrounds in the future.

Plant transformation involves the construction of an expression vector which will function in plant cells. Such a vector is comprised of DNA involving a gene under the control of, or operatively linked to, a regulatory element (for example, a promoter). The expression vector may contain one or more such operatively linked gene/regulatory element combinations. The vector(s) may be in the form of a plasmid, and can be used alone or in combination with other plasmids, to provide transformed Brassica plants, using transformation methods as described below to incorporate transgenes into the genetic material of the Brassica plant(s).

Expression Vectors for Brassica Transformation: Marker Genes—Expression vectors include at least one genetic marker, operatively linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or a herbicide, or genes that encode an altered target which is insensitive to the inhibitor. A few positive selection methods are also known in the art.

Various promoters can be used to make the gene constructs, which in turn can be used for transformation. Several types of promoters are now well known in the transformation arts, as are other regulatory elements that can be used alone or in combination with promoters. These promoters can include but not limited to promoters of constitutive, inducible, tissue specific and organ specific etc, which are well described in the arts.

Likewise, by means of the present invention, agronomic genes can be expressed in transformed plants. Specifically, the inbred lines disclosed in the current invention can be used for transformation to express various genes of interests. Exemplary genes implicated in this regard include, but are not limited to, those categorized below:

1. Genes that Confer Resistance to Pests or Disease and that Encode:

A. Plant disease resistance genes. Plant defences are often activated by specific interactions between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example Jones et al. (Science 266:789, 1994); Martin et al. (Science 262:1432, 1993); Mindrinos et al. (Cell 78:1089, 1994).

B. A gene conferring resistance to a pest, such as soybean cyst nematode. See for example WO 96/30517; WO 93/19181.

C. A Bacillus thuringiensis protein, a derivative thereof or a synthetic polypeptide modeled thereon. See, for example, Geiser et al., Gene 48:109, 1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

D. A lectin. See, for example, the disclosure by Van Damme et al. (Plant Molec. Biol. 24:25, 1994), who disclose the nucleotide sequences of several Clivia miniata mannose-binding lectin genes.

E. A vitamin-binding protein such as avidin (see US93/06487). The application teaches the use of avidin and avidin homologues as larvicides against insect pests.

F. An enzyme inhibitor, for example, a protease or proteinase inhibitor or an amylase inhibitor. See, for example, Abe et al. (J. Biol. Chem. 262:16793, 1987), Huub et al. (Plant Molec. Biol. 21:985, 1993), Sumitani et al. (Biosci. Biotech. Biochem. 57:1243, 1993), and U.S. Pat. No. 5,494,813.

G. An insect-specific hormone or pheromone such as an ecdysteroid or juvenile hormone, a variant thereof, a mimetic based thereon, or an antagonist or agonist thereof. See, for example, the disclosure by Hammock et al. (Nature 344:458, 1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. (Biol. Chem. 269:9, 1994), Pratt et al. (Biochem. Biophys. Res. Comm 163:1243, 1989) and U.S. Pat. No. 5,266,317 (disclosing genes encoding insect-specific, paralytic neurotoxins).

I. An insect-specific venom produced in nature by a snake, a wasp, etc. For example, see Pang et al. (Gene 116:165, 1992), for disclosure of heterologous expression in plants of a gene coding for a scorpion insectotoxic peptide.

J. An enzyme responsible for a hyper-accumulation of a monoterpene, a sesquiterpene, a steroid, a hydroxamic acid, a phenylpropanoid derivative or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See WO 93/02197 which discloses the nucleotide sequence of a callase gene. DNA molecules which contain chitinase-encoding sequences can be obtained, for example, from the ATCC under Accession Nos. 39637 and 67152. See also Kramer et al. (Insect Biochem. Molec. Biol. 23:691, 1993), who teach the nucleotide sequence of a cDNA encoding tobacco hornworm chitinase, and Kawalleck et al. (Plant Molec. Biol. 21:673, 1993), who provide the nucleotide sequence of the parsley ubi4-2 polyubiquitin gene.

L. A molecule that stimulates signal transduction. For example, see the disclosure by Botella et al. (Plant Molec. Biol. 24:757, 1994), of nucleotide sequences for mung bean calmodulin cDNA clones, and Griess et al. (Plant Physiol. 104:1467, 1994), who provide the nucleotide sequence of a maize calmodulin cDNA clone.

M. A hydrophobic moment peptide. See WO 95/16776 (disclosure of peptide derivatives of Tachyplesin which inhibit fungal plant pathogens) and WO 95/18855 (teaches synthetic antimicrobial peptides that confer disease resistance).

N. A membrane permease, a channel former or a channel blocker. For example, see the disclosure of Jaynes et al. (Plant Sci 89:43, 1993), of heterologous expression of a cecropin-β, lytic peptide analog to render transgenic tobacco plants resistant to Pseudomonas solanacearum.

O. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al. (Ann. Rev. Phytopathol. 28:451, 1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus.

P. An insect-specific antibody or an immunotoxin derived from. Thus, an antibody targeted to a critical metabolic function in the insect gut would inactivate an affected enzyme, killing the insect. Cf. Taylor et al. (Abstract #497, Seventh Int'l Symposium on Molecular Plant-Microbe Interactions; Edinburgh, Scotland, 1994, “Enzymatic inactivation in transgenic tobacco via production of single-chain antibody fragments”).

Q. A virus-specific antibody. See, for example, Tavladoraki et al. (Nature 366:469, 1993), who show that transgenic plants expressing recombinant antibody genes are protected from virus attack.

R. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb et al. (Bio/Technology 10:1436, 1992). The cloning and characterization of a gene which encodes a bean endopolygalacturonase-inhibiting protein is described by Toubart et al. (Plant J. 2:367, 1992).

S. A developmental-arrestive protein produced in nature by a plant. For example, Logemann et al. (Bio/Technology 10:305, 1992) have shown that transgenic plants expressing the barley ribosome-inactivating gene have an increased resistance to fungal disease.

2. Genes that Confer Resistance to Herbicides:

A. An herbicide that inhibits the growing point or meristem, such as an imidazolinone or a sulfonylurea. Exemplary genes in this category code for mutant ALS and AHAS enzyme as described, for example, by Lee et al. (EMBO J. 7:1241, 1988), and Mild et al. (Theor. Appl. Genet. 80:449, 1990), respectively.

B. Glyphosate (resistance conferred by mutant 5-enolpyruvyl-shikimate-3-phosphate synthase (EPSP) and aroA genes, respectively) and other phosphono compounds such as glufosinate (phosphinothricin acetyl transferase (PAT) and Streptomyces hygroscopicus PAT, bar, genes), and pyridinoxy or phenoxy proprionic acids and cyclohexones (ACCase inhibitor-encoding genes). See, for example, U.S. Pat. No. 4,940,835 which discloses the nucleotide sequence of a form of EPSP which can confer glyphosate resistance. A DNA molecule encoding a mutant aroA gene can be obtained under ATCC accession number 39256, and the nucleotide sequence of the mutant gene is disclosed in U.S. Pat. No. 4,769,061. European patent application No. 0 333 033, and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246. DeGreef et al. (Bio/Technology 7:61, 1989), describe the production of transgenic plants that express chimeric bar genes coding for PAT activity. Exemplary of genes conferring resistance to phenoxy proprionic acids and cyclohexones, such as sethoxydim and haloxyfop are the Acc1-S1, Acc1-S2 and Acc1-S3 genes described by Marshall et al. (Theor. Appl. Genet. 83:435, 1992).

C. An herbicide that inhibits photosynthesis, such as a triazine (psbA and gs+ genes) or a benzonitrile (nitrilase gene). Przibila et al. (Plant Cell 3:169, 1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al. (Biochem. J. 285:173, 1992).

3. Genes that Confer or Contribute to a Value-Added Trait, Such as:

A. Modified fatty acid metabolism, for example, by transforming a plant with an antisense gene of stearyl-ACP desaturase to increase stearic acid content of the plant. See Knultzon et al. (Proc. Natl. Acad. Sci. U.S.A. 89:2624, 1992).

B. Decreased phytate content—1) Introduction of a phytase-encoding gene would enhance breakdown of phytate, adding more free phosphate to the transformed plant. For example, see Van Hartingsveldt et al. (Gene 127:87, 1993), for a disclosure of the nucleotide sequence of an Aspergillus niger phytase gene. 2) A gene could be introduced that reduced phytate content. In maize, this, for example, could be accomplished, by cloning and then reintroducing DNA associated with the single allele which is responsible for maize mutants characterized by low levels of phytic acid. See Raboy et al. (Maydica 35:383, 1990).

C. Modified carbohydrate composition effected, for example, by transforming plants with a gene coding for an enzyme that alters the branching pattern of starch. See Shiroza et al. (J. Bacteol. 170:810, 1988) (nucleotide sequence of Streptococcus mutants fructosyltransferase gene), Steinmetz et al. (Mol. Gen. Genet. 20:220, 1985) (nucleotide sequence of Bacillus subtilis levansucrase gene), Pen et al. (Bio/Technology 10:292, 1992), Elliot et al. (Plant Molec. Biol. 21:515, 1993), SØgaard et al. (J. Biol. Chem. 268:22480, 1993), and Fisher et al. (Plant Physiol. 102:1045, 1993).

The present invention will be further illustrated in the following examples.

### Example 1

### Development of Canola Quality B. juncea CMS (Mori) Lines

A cytoplasmic male sterility mustard (Brassica juncea) line, MoriS, was obtained in June 2007 from Dr. Prakash of the National Research Centre on Plant Biotechnology, Indian Agricultural Research Institute (New Delhi 110012, India). MoriS was initially developed with a cross between Moricandia arvensis and a mustard juncea (variety Pusa Bold') with a goal of placing the mustard juncea nucleus in the cytoplasm of wild species Moricandia arvensis (Prakash et al., Theor Appl Genet, 1998, 97: 488-492). After the initial cross, Pusa Bold was repeatedly used as the recurrent parent for backcrossing.

The development of canola-quality Brassica juncea CMS (mori) lines began June 2007. MoriS was used as the CMS trait donor and was crossed to adapted, canola-quality Brassica juncea lines with subsequent repeated backcrossing to several canola-quality Brassica juncea varieties and breeding lines through 2010. As shown in Table 1, both MoriS and the F1 of the initial cross, XJ07-084, had high levels of total glucosinolates and erucic acid and low oleic acid. With continued backcrossing to canola quality B. juncea, and selection, significant improvements in quality were observed. After multiple crosses and backcrosses, AM-J05Z-10367(3) is used as CMS (mori) line with canola quality (Table 1). In addition, AM-J05Z-10367(3) has a total saturated fatty acids of 6.95%. Seeds harvested of AM-J05Z-10367(3) were named AM-J05Z-10367 and deposited with the ATCC (ATCC Accession No. PTA-12261, Nov. 17, 2011).

TABLE 1
Quality Analysis of Lines and Crosses during Development of Canola
Quality B. juncea CMS (mori) lines. MoriS is the mustard juncea
used as CMS (mori) trait donor. Variety Estlin and breeding line
J05Z-10367 are both canola quality used as nucleus genome donor. Both
total glucosinolates and fatty acids are analyzed by GLC as described.
The numbers in bracket after the line AM-J05Z-10367 denote generations
of backcrosses with line J05Z-10367. AM-J05Z-10367 (3) was used for
making hybrids, and renamed AM-J05Z-10367.

| | | Total | | |
|---|---|---|---|---|
| | | Glucosinolates | | |
| | | (μmole/g oil | Erucic | Oleic |
| Name | Pedigree | free dry meal) | Acid % | Acid % |
| MoriS | | 91.22 | 44.66 | 8.43 |
| Estlin | | 9.64 | 0.00 | 61.10 |
| J05Z-10367 | | 12.31 | 0.04 | 63.08 |
| XJ07-084 | MoriS/Estlin | 156.75 | 31.44 | 16.78 |
| XJ07-157 | MoriS/2*Estlin | 92.04 | 17.37 | 33.85 |
| AM-Estlin | MoriS/3*Estlin | 52.96 | 0.00 | 47.68 |
| AM-J05Z- | MoriS/4*Estlin// | 32.75 | 0.00 | 55.25 |
| 10367 (1) | J05Z-10367 | | | |
| AM-J05Z- | MoriS/4*Estlin// | 22.51 | 0.00 | 64.60 |
| 10367 (2) | 2*J05Z-10367 | | | |
| AM-J05Z- | MoriS/4*Estlin// | 7.95 | 0.00 | 59.74 |
| 10367 (3) | 3*J05Z-10367 | | | |

Many other CMS (mori) B. juncea lines with canola quality were developed with similar methods. For example, AM-J05Z-09677 and AM-J05Z-08376 were developed using same backcrossing methods with canola quality B. juncea lines J05Z-09677 and J05Z-08376, respectively. Other available CMS (mori) B. juncea lines with canola quality include AM-J06Z-02993, AM-J07Z-04703, AM-J07Z-08478 and AM-J07Z-09190 (from Viterra Inc, Saskatoon Sask Canada). The development of multiple CMS (mori) B. juncea lines with canola quality made it possible to test many hybrids with different genetic backgrounds.

### Example 2

### Development of the Canola Quality B. juncea Fertility Restorer Lines

A fertility restorer mustard (Brassica juncea) line, MoriR4, was also obtained in June 2007 from Dr. Prakash of the National Research Centre on Plant Biotechnology, Indian Agricultural Research Institute (New Delhi 110012, India). MoriR4 was initially developed from a cross between Moricandia arvensis and a mustard juncea (variety ‘Pusa Bold’). After backcrossing repeatedly to mustard juncea, an individual plant in the third backcross generation (BC3) with 18% pollen fertility was identified (Prakash et al., Theor Appl Genet, 1998, 97: 488-492). This individual plant was backcrossed a few more times to mustard juncea, thus increasing the fertility rate to 93% in the BC6 generation. Therefore, a plant with a fertility rate for from 50-100% or any amount therebetween may be used as described herein.

The development of canola-quality Brassica juncea restorer lines began in June 2007. MoriR4 was used as the fertility restorer gene donor and was crossed to adapted, canola-quality Brassica juncea lines with repeated backcrosses to several canola-quality Brassica juncea varieties and breeding lines through 2010. As shown in Table 2, MoriR4 and the F1 of the initial crosses, XZ07-0128 and XZ07-162, all have high levels of total glucosinolates, high erucic acid and low oleic acid. With continued backcrossing to canola quality B. juncea, significant improvements in quality were achieved. After canola quality was achieved, crosses XZ0-8124 and XZ0-8183 were used as donors to produce doubled haploid (DH) lines. DH lines have homozygous gene alleles including the fertility restorer gene (Rfm), which is very important for making the hybrid and evaluating the level of hybridity. Individual DH lines were evaluated and analyzed for quality. Many canola quality B. juncea fertility restorer (Rfm) DH lines were produced. Listed in table 2, JM0Z-907526, JM0Z-907555 and JM0Z-909643 are a few representative lines from these groups. Seeds of JM0Z-909643 were deposited with the ATCC (ATCC Accession No. PTA-12260, Nov. 17, 2011).

TABLE 2
Quality Analysis of Lines and Crosses during Development of Canola
Quality B. juncea Restorer Lines. MoriR4 is the mustard juncea
used as the restorer gene donor. Variety Estlin and breeding
line J05Z-07784 are canola quality. Both total glucosinolates
and fatty acids are analyzed by GLC as described.

| | | Total | | |
|---|---|---|---|---|
| | | Glucosinolates | | |
| | | (μmole/g oil | Erucic | Oleic |
| Name | Pedigree | free dry meal) | Acid % | Acid % |
| MoriR4 | | 91.50 | 47.55 | 6.75 |
| Estlin | | 9.64 | 0.00 | 61.10 |
| J05Z- | | 13.24 | 0.05 | 56.91 |
| 07784 | | | | |
| XZ07-128 | J05Z-07784/MoriR4 | 66.3 | 31.44 | 16.78 |
| XZ07-162 | XZ07-128/J05Z-07784 | 59.10 | 21.80 | 27.40 |
| XZ0-8050 | XZ07-162/J05Z-07784 | 16.10 | 20.20 | 32.70 |
| XZ0-8124 | XZ0-8050/J05Z-07784 | 23.2 | 0-.00 | 58.10 |
| JM0Z- | (DH from XZ0-8124 | 11.50 | 0.12 | 61.13 |
| 907526 | as donor) | | | |
| JM0Z- | (DH from XZ0-8124 | 10.60 | 0.11 | 63.13 |
| 907555 | as donor) | | | |
| JM0Z- | (DH from XZ0-8183 | 12.13 | 0.05 | 64.53 |
| 909643 | as donor) | | | |

### Example 3

### Moricandia Genome Fragment Sequence-Specific Marker Development

The chromosome location of the Moricandia arvensis fragment flanking the fertility restorer gene for the mori CMS has not been determined. A SCAR marker was available for the mustard juncea, which produced two amplifications for the fertility restorer lines when analyzed by gel electrophoresis (Ashutosh et al., 2007, Theor Appl Genet 114: 385-392). When we tested this marker on canola quality B. juncea fertility restorer (mori) lines, one amplification band was missing from the gel. To make sure this amplification is indeed associated with the Rfm gene, we cloned the amplification product and sequenced the complete insert, which produced a 187 bp unique sequence as shown in FIG. 1.

A BLAST search using the 187 bp insert sequence as a query indicated that this insert sequence does not have high similarity with any known sequence in the public database (see URL: ncbi.nlm.nih.gov). Insert sequence specific primers, RfmF (5′-TCACTAAAGATCGAGATAGTACCA-3′; SEQ ID NO:1) and RfmR (5′-TAACATCTTCAACGTTTCGGTG; SEQ ID NO:2), were designed and tested in PCR screening. The PCR was performed in a total volume of 25 ul, which contained 0.5 uM each of the primers, 20 ng of genomic DNA, 1 mM each of dATP, dCTP, dGTP and dTTP, 50 mM KCl, 10 mM Tris pH 8.3, 1.5 mM MgCl₂ and 1 unit of Taq polymerase. The DNA template was denatured for 5 mM at 94° C. before amplification. The amplification was done with 32 cycles of 1 mM at 94° C., 40 sec at 61° C. and 1 mM at 72° C. The final PCR mixture was incubated at 72° C. for 10 mM after cycling.

The amplification product of 187 bp fragment, which appeared on 1.4% agarose gel as a single band after electrophoresis, was used to determine the presence of the Rfm gene. The primer pair works very well for screening the presence of the Rfm gene using DNA from tissues such as leaf and seeds. This new marker is more accurate and more efficient.

The primers have been used in PCR screening and all marker positive samples have produced viable pollen in the greenhouse and field. No individual DH plant has been found so far that has a recombination between the marker and the Rfm gene.

### Example 4

### Hybrid Seed Production and Hybridity Test

Many experimental hybrid lines were made by combining multiple canola quality CMS (mori) lines and several canola quality fertility restorer lines. These experiment hybrids were made by growing one or more CMS lines with only one restorer line in a self contained mini tent. Flies were used to facilitate the process of pollination. Hybrid seeds were harvested from the CMS plant only.

Harvested hybrid seeds were also analyzed for quality. All hybrid seeds were canola quality in terms of total glucosinolates, erucic acid and oleic acid content. This confirmed that the quality of hybrid seed is mostly controlled by the quality of inbred lines (parents of hybrids). When both the CMS line and the fertility restorer line are canola quality, the hybrid made from the combination is expected to be canola quality.

Many experimental hybrid lines were made in an isolated open field in Chile which simulates the commercial hybrid seed production situation. These experimental hybrid seeds were harvested from CMS lines. All hybrids were made from the restorer line JM0Z-909643 (ATCC accession No. PTA-12260, Nov. 17, 2011) with different CMS lines. Levels of hybridity were determined by checking if the hybrid seeds have the Rfm gene. A minimum of 48 individual seeds were tested for each hybrid line. As shown in Table 3, the level of hybridity varied from 90% to 100%. However, a hybridity of from 60-100% or any amount therebetween may be used as described herein. The hybrid HJM1Z-1162, made from CMS line AM-J05Z-10367 (ATCC accession No. PTA-12261, Nov. 17, 2011) and the restorer line JM0Z-909643 (ATCC accession No. PTA-12260, Nov. 17, 2011), has a level of hybridity of 97.5%.

TABLE 3
Hybridity levels of B. juncea hybrid lines. The hybridity
level was determined by the presence of Rfm marker gene in the
hybrid seeds, which is expressed as the percentage of number
of seeds that are positive for Rfm marker gene over the total
of seeds tested. All hybrid seed lines were made using various
mori CMS lines with the single restorer line JM0Z-909643. HJM1Z-1162
was made using mori CMS line AM-J05Z-10367.

| Hybrid Lines | CMS (mori) Line | Hybridity Level |
|---|---|---|
| HJM1Z-1010 | AM-J05Z-09677 | 90.0% |
| HJM1Z-1161 | AM-J05Z-08376 | 92.5% |
| HJM1Z-1162 | AM-J05Z-10367 | 97.5% |
| HJM1Z-1163 | AM-J06Z-02993 | 95.0% |
| HJM1Z-1166 | AM-J07Z-04703 | 100% |
| HJM1Z-1167 | AM-J05Z-08478 | 97.2% |
| HJM1Z-1168 | AM-J05Z-09190 | 94.4% |

### Example 5

### Yield Potential of Hybrid Seeds

Experimental mori canola quality B. juncea hybrids were tested in yield trials in 6 locations across western Canada in 2011. Elite open-pollinated canola quality B. juncea varieties were included in the trial as reference varieties. The data collected from these trials included seed quality analyses and yield (kg/ha). As shown in Table 4, selected hybrids from this trial met the quality standards as defined for canola quality varieties in terms of glucosinolate content (<30 μmole/g oil free dry meal), erucic acid (<2% by weight) and oleic acid (>55% by weight). As well, the selected hybrid lines yielded from 104% to 108% in comparison with elite open pollinated varieties. This clearly indicates that the mori CMS hybrid system is a viable system for producing canola quality B. juncea hybrid varieties as well as for improving yield potential vs. open-pollinated varieties. This proves that a canola quality B. juncea hybrid variety can be produced with the mori CMS hybrid system as disclosed in the current invention. This new hybrid system provides a valuable tool for increasing yield of canola quality B. juncea. The yield advantage demonstrated in this document is significant as these experimental hybrids represent only the first cycle of experimental B. juncea hybrids whereas the reference varieties have been bred extensively for yield for over 20 years.

Initial hybrid yield tests involve multiple CMS lines and restorer lines (see Table 4). The specific hybrid HJM1Z-1162 was not selected for yield test because both CMS line AM-J05Z-10367 (ATCC accession No. PTA-12261, Nov. 17, 2011) and the restorer line JM0Z-909643 (ATCC accession No. PTA-12260, Nov. 17, 2011), have the same genetic background of line J05Z-10367. Within such a narrow genetic diversity, the heterosis of hybrid HJM1Z-1162 would not be high.

With the mori CMS hybrid system described herein, increase heterosis with more diversified genetic backgrounds should be within routine plant breeding practice. Therefore, further increases in heterosis can be achieved as new diversified genetic backgrounds are explored in the future.

TABLE 4
Yield Comparison of B. juncea Hybrid Lines vs. Open Pollinated
Varieties (Oasis CL and Xceed 8571). The selected hybrid lines were
made with a combination of different CMS lines and restorer lines
including the lines described herein. Yield potential was expressed
as a percentage of the average of the two open pollinated varieties.
Data was collected in summer 2011 from field grown hybrid seeds and
is expressed as an average of 6 locations in western Canada.

| | Glucosinolates | Erucic | Oleic | Relative |
|---|---|---|---|---|
| Hybrid Lines | (μmole/g) | Acid % | Acid % | Yield % |
| Oasis CL and | 15.0 | 0.24 | 63.1 | 100 |
| Xceed 8571* | | | | |
| HJM1Z-1001 | 25.0 | 0.53 | 63.3 | 106.6 |
| HJM1Z-1002 | 17.4 | 0.35 | 64.3 | 103.6 |
| HJM1Z-1006 | 16.6 | 0.30 | 64.8 | 107.6 |
| HJM1Z-1010 | 15.2 | 0.27 | 65.5 | 107.3 |
| HJM1Z-1014 | 19.1 | 0.3 | 65.1 | 105.4 |
| HJM1Z-1029 | 22.7 | 0.39 | 64.3 | 106.6 |
| HJM1Z-1034 | 15.0 | 0.37 | 65.2 | 107.6 |

*Oasis CL and Xceed 8571 are the current canola quality open pollinated B. juncea varieties grown in western Canada.

Indeed, in 2012 summer field trials, newly created B. juncea hybrid lines were tested, along with the two hybrid lines tested in year 2011. The yield results are summarized in Table 5. All the newly created 5 hybrid lines (HJM1Z-2056, HJM1Z-2138, HJM1Z-2142, HJM1Z-2080 and HJM1Z-2065) were made from various A lines with the same restorer line, JM0Z-90618, that was used to make hybrid line HJM1Z-1006. In comparison, 4 of the 5 newly created hybrid lines (HJM1Z-2056, HJM1Z-2138, HJM1Z-2142, HJM1Z-2080) show significant improvement in heterosis (Table 5).

TABLE 5
Yield Comparison of B. juncea Hybrid Lines vs. Open Pollinated
Varieties (Oasis CL and Xceed 8571). The selected hybrid lines
were made with a combination of different CMS lines and restorer
lines. Yield potential was expressed as a percentage of the average
of the two open pollinated varieties. Data was collected in summer
2012 from field grown hybrid seeds and is expressed as an average
of 5 locations in western Canada.

| | Glucosinolates | Erucic | Oleic | Relative |
|---|---|---|---|---|
| Hybrid Lines | (μmole/g) | Acid % | Acid % | Yield % |
| Oasis CL and | 14.0 | 0.19 | 61.9 | 100 |
| Xceed 8571* | | | | |
| HJM1Z-1006 | 16.6 | 0.30 | 64.8 | 105 |
| HJM1Z-1034 | 15.0 | 0.37 | 65.2 | 117 |
| HJM1Z-2056 | 15.0 | 0.17 | 63.1 | 118 |
| HJM1Z-2138 | 18.3 | 0.06 | 63.9 | 122 |
| HJM1Z-2142 | 18.3 | 0.00 | 64.4 | 125 |
| HJM1Z-2080 | 15.3 | 0.17 | 64.2 | 117 |
| HJM1Z-2065 | 15.1 | 0.44 | 63.8 | 108 |

*Oasis CL and Xceed 8571 are the current canola quality open pollinated B. juncea varieties grown in western Canada. Hybrid lines HJM1Z-1006 and HJM1Z-1034 that were tested in 2011 (Table 4) and are re-tested in 2012. All other hybrid lines are made from various A lines with the same restorer line, JM0Z-90618, that was used to make HJM1Z-1006.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A crushed seed meal-oil composition obtained by crushing a seed from Brassica juncea oil seed plant comprising cytoplasm of Moricandia arvensis, the Brassica juncea oil seed plant characterized as being cytoplasmic male sterile, and the seed comprising canola quality oil and canola quality meal.

2. The crushed seed meal-oil composition of claim 1, wherein the seed comprises less than 30 umole per gram oil-free total glucosinolates.

3. The crushed seed meal-oil composition of claim 1, wherein the seed comprises less than 2% erucic acid by weight.

4. The crushed seed meal-oil composition of claim 1, wherein the seed comprises more than 55% oleic acid by weight.

5. The crushed seed meal-oil composition of claim 1, wherein the seed is obtained from progeny obtained from a cross of Brassica juncea line AM-J05Z-10367 that has been deposited and has a ATCC accession No. PTA-12261 with a canola quality B. juncea line.

6. A plant cell derived from a Brassica juncea plant that produces the crushed seed meal-oil composition of claim 1.

7. A plant cell derived from a Brassica juncea oilseed plant deposited under ATCC accession No. PTA-12260.

8. A plant cell derived from a Brassica juncea oilseed plant deposited under ATCC accession No. PTA-12261.

9. A method of making hybrid Brassica juncea seed comprising, crossing a first Brassica juncea oil seed plant comprising cytoplasm of Moricandia arvensis, the first B. juncea oil seed plant characterized as being cytoplasmic male sterile, the seed obtained from the first plant comprising canola quality oil and canola quality meal with a second B. juncea oilseed plant comprising a homozygous fertility restorer gene (Rfm) for mori cytoplasmic male sterility, the seed obtained from the second plant have an oil and meal in canola quality, to produce the hybrid B. juncea seed, the hybrid Brassica juncea seed characterized as canola quality and comprising a hybridity level of at least of 90%.

10. The method of claim 9, wherein, wherein the hybrid Brassica juncea seed comprises less than 30 umole per gram oil-free total glucosinolates, less than 2% erucic acid by weight, and more than 55% oleic acid by weight.

11. A hybrid Brassica juncea plant cell obtained from a hybrid Brassica juncea plant grown from the hybrid Brassica juncea seed produced by the method of claims 10.

12-13. (canceled)

14. A method of obtaining an oil comprising less than 30 umole per gram oil-free total glucosinolates, less than 2% erucic acid by weight, and more than 55% oleic acid by weight, the method comprising:
growing a plant produced from a seed from a cytoplasmic male sterile Brassica juncea oil seed plant comprising cytoplasm of Moricandia arvensis;
obtaining progeny seed from the plant; and
extracting the oil from the progeny seed obtained from the plant.

15. A method of obtaining an oil comprising less than 30 umole per gram oil-free total glucosinolates, less than 2% erucic acid by weight, and more than 55% oleic acid by weight, the method comprising, extracting the oil from the hybrid Brassica juncea seed obtained using the method of claim 10.
